(19) United States
(12) Patent Application Publication
(10) Pub. No.: US 2003/0186426 A1
(43) Pub. Date: Oct. 2, 2003

(54) Multichannel flow cell for interacting single optically trapped, DNA molecules with different chemical species

(75) Inventors: Laurence R. Brewer, Oakland, CA (US); Rodney L. Balhorn, Livermore, CA (US)

Correspondence Address: Alan H. Thompson, Assistant Laboratory Counsel, Lawrence Livermore National Laboratory, P.O. Box 808, L-703, Livermore, CA, 94551, US

(73) Assignee: The Regents of the University of California

(21) Appl. No.: 09/809,944

(22) Filed: Mar. 14, 2001

Related U.S. Application Data
(60) Provisional application No. 60/189,381, filed on Mar. 15, 2000.

(51) Int. Cl. C12M 1/34

(52) U.S. Cl. 435/287.2

(57) ABSTRACT

A multichannel flow cell is used to laminarly flow different chemical solutions, including one made up of small polystyrene beads attached to individual DNA molecules, side by side with little mixing. An optical trap is used to pull single DNA molecules via their attached polystyrene beads into each of the different chemical solutions or species sequentially, and the resultant change in the structure of the DNA molecule can be observed using fluorescence microscopy. The technique can be used with molecules other than DNA. Examples of different chemical species include condensing agents such as protamine, enzymes, polymerases, and fluorescent probes and tages.

### RELATED APPLICATION

This application relates to U.S. Provisional Application No. 60/189,381, filed Mar. 15, 2000, and claims priority thereof.

The United States Government has rights in this invention pursuant to Contract No. W-7405-ENG-48 between the United States Department of Energy and the University of California for the operation of Lawrence Livermore National Laboratory.

### BACKGROUND OF THE INVENTION

The present invention relates to the manipulation of molecules, particularly to the manipulation of individual DNA molecules in laminar flow, and more particularly to the manipulation of molecules using a multichannel flow cell and an optical trap for interacting single, optically trapped, DNA molecules in a laminar flow of different chemical solutions side by side with little mixing. Methods and various apparatus for the manipulation of molecules in a laminar flow, particularly the manipulation of individual DNA molecules, have been developed. One approach to manipulation of molecules and particles has involved optical trapping, as exemplified by U.S. Pat. No. 5,079,169 issued Jan. 7, 1992; U.S. Pat. No. 5,495,105 issued Feb. 27, 1996; U.S. Pat. No. 5,620,857 issued Apr. 15, 1997; and U.S. Pat. No. 5,952,651 issued Sep. 14, 1999.

The ability to introduce an individual DNA molecule to proteins or a variety of different chemical environments both at a precise time, and sequentially, is essential for studies that characterize the binding of proteins to DNA. One prior approach has been to use a multiport valve on a single channel flow cell. However, this approach is problematic. Pressure surges introduced by changing the valve setting often move the bead attached to the DNA molecule from the optical trap. Also, flow speeds must not be too fast or the trapped bead is dislodged. This means that it can take a long time to replace one chemical species in the flow cell with another chemical species. In addition, there is a high probability of losing the trapped bead through direct collision with another bead (from the original DNA-bead sample) during the introduction of a new chemical species via the multiport valve into the single flow channel. Thus, there has been a need for a more effective way to introduce a DNA molecule, for example, to a variety of different chemical environments.

The present invention provides a solution to that need by providing an alternative approach using a plural channel or multiple channel multiple flow cell where different chemical species are introduced into the flow cell simultaneous. The multichannel flow cell of this invention enables interacting single, optically trapped, DNA or other molecules with different chemical species. The multichannel flow cell of the present invention is used to flow different chemical solutions in a laminar manner side by side with little mixing. An optical trap is used, for example, to pull single DNA molecules via their attached polystyrene beads in to each of the different chemical species sequentially, and the resultant change in the structure of the DNA molecule can be observed using fluorescence microscopy.

### SUMMARY OF THE INVENTION

It is an object of the present invention to sequentially introduce an individual molecule to a variety of different chemical environments.

A further object of the invention is to enable studies that characterize the binding of proteins, for example, to DNA.

A further object of the invention is to provide a multichannel flow cell used to laminarly flow different chemical solution side by side with little mixing.

Another object of the invention is to provide a multichannel flow cell for interacting, optically trapped, DNA molecules with different chemical species.

Another object of the invention is to provide a multichannel flow cell with an optical trapping arrangement, wherein molecules such as individual DNA molecules attached to small polystyrene beads can be moved sequentially into different channels containing different chemical species, and the resultant change in the structure of the DNA molecule can be observed using fluorescence microscopy and/or force molecules via the optical trap.

Other objects and advantages of the present invention will become apparent from the following description and accompanying drawings. The present invention involves a multichannel flow cell used to laminarly flow different chemical solutions side by side with little mixing. The flow cell utilizes an optical trap to pull single molecules, such as one made up of small polystyrene beads attached to individual molecules, sequentially into each of the different channels containing different chemical species, whereby the resultant change in the structure of the DNA molecule can be observed using fluorescence microscopy. This approach can be used with molecules other than DNA. This allows one to study the interaction between single DNA molecules and any chemical species and to examine the structural changes in the DNA molecule. The invention can be used to study different condensing agents for packaging DNA for gene therapy, sequentially binding a variety of molecules to single molecules or characterizing how an ordered assembly of molecules affects the final structure of a macromolecular complex.

### BRIEF DESCRIPTION OF THE DRAWINGS

The accompanying drawings, which are incorporated into and form a part of the disclosure, illustrate embodiments of the invention and, together with the description, serve to explain the principles of the invention.

FIG. 1A is an exploded view of an embodiment of a multichannel flow cell made in accordance with the present invention.

FIG. 1B illustrates an enlarged portion from within the circled area of FIG. 1A showing DNA molecules with attached beads and an individual DNA molecule held in place by an optical trap, with the dashed lines representing interfaces between the liquids in the multichannels of the flow cell.

FIG. 2 is a top view of a bifurcated flow cell utilized to experimentally verify the invention.

FIG. 3 is a view of an infrared optical trap used to move an individual DNA molecule, via its attached bead, from the sample (DNA) side to the condensing agent (protein) side of the flow cell of FIG. 2.

FIG. 4 graphically illustrates the change in length verses time for four different DNA molecules as they condensed in different concentrations of protamine.

FIG. 5 graphically illustrates experiments conducted at different protamine concentrations which shows that the rate of condensation was limited by the rate of protamine binding to the DNA module, and that the change in rate was linear.

### DETAILED DESCRIPTION OF THE INVENTION

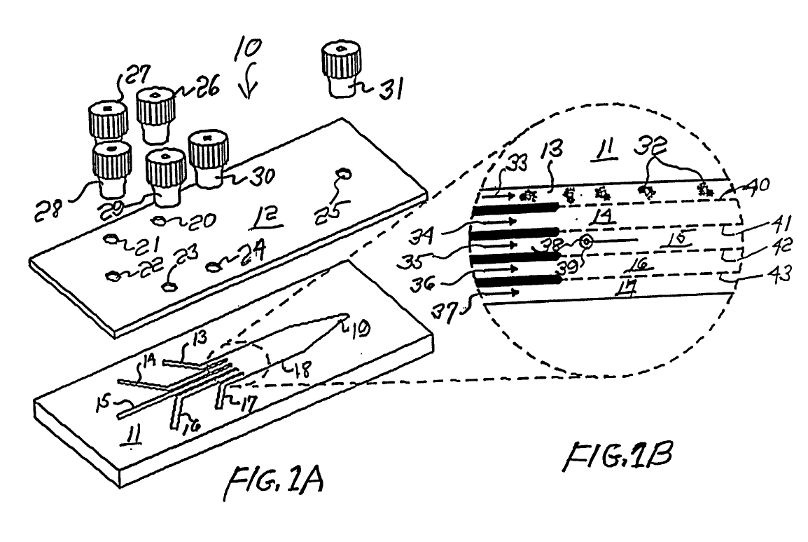

The present invention is directed to a multichannel flow cell which provides the ability to introduce an individual DNA molecule to proteins or a variety of different chemical environments both at a precise time, and sequentially, thus enabling studies that characterize the binding of proteins to DNA. The multichannel/multiport flow cell of this invention overcomes the above-referenced problems associated with the prior known multiport single channel flow cell. With the multichannel flow cell of the present invention different chemical species may be introduced into the flow cell simultaneously, as seen in FIGS. 1A and 1B.

The multichannel, multiport flow cell, as shown in FIGS. 1A and 1B, generally indicated a 10, comprises a lower plate or slide 11 and an upper plate or slide 12. Lower plate 11 has multiple input channels sections 13, 14, 15, 16 and 17 which are directed into a common channel 18 having a tapered output section 19, while upper plate 12 is provided with a plurality of holes or openings 20, 21, 22, 23, 24 and 25 which align with channels 13-17 and 19, and into which ports or connectors 26, 27, 28, 29, 30 and 31 are mounted. FIG. 1B illustrates an enlarged portion of input channel sections 13-17 and common channel 18 defined by the circled area of lower plate or slide 11. DNA molecules with attached beads indicated at 32 are introduced into the port 26, opening 20 and top most channel section 13 as indicated by arrow 33, and four (4) other proteins/peptides are introduced into the remaining ports 27-30, opening 21-25 and input channel sections 14-17, as indicated by arrows 34, 35, 36 and 37. An individual DNA molecule including a bead 38 is held in place by an optical trap indicated by the circle 39 around bead 38 in the channel section 15 containing a second protein solution. The dashed lines 40, 41, 42 and 43 represent interfaces between the liquids in respective channels 13-17. The optical trap 39 may be of the type shown in FIG. 3, described hereinafter or by any of the above-referenced patents.

The flow of the different chemical species via input channel section 13-17 is laminar at Reynolds numbers Re<2000 (37), where

Rₑ=vlp/η

(v, the fluid velocity, 1, the microchannel depth, ρ the fluid density, and η the fluid viscosity are all in MKS units). For typical flow cell conditions, v=100 um/sec, ρ=1.23 gm/sec, 1=40 um, and η=15.3 cp, we find (after converting to MKS units) that Rₑ=3.3, amply satisfying the above criterion. The trapped DNA molecule 38 can then be rapidly placed in contact with a different chemical species by moving a stage containing the flow cell transversely to the direction of flow. Assuming the widths of the different channels are 1 mm, it would take 20 seconds to cross one, moving the stage holding the flow cell at a speed of 50 um/sec. Thus, a DNA molecule could be introduced to three different chemical species fairly quickly, moving from the center of the first channel, to the center of the third, in approximately 40 seconds.

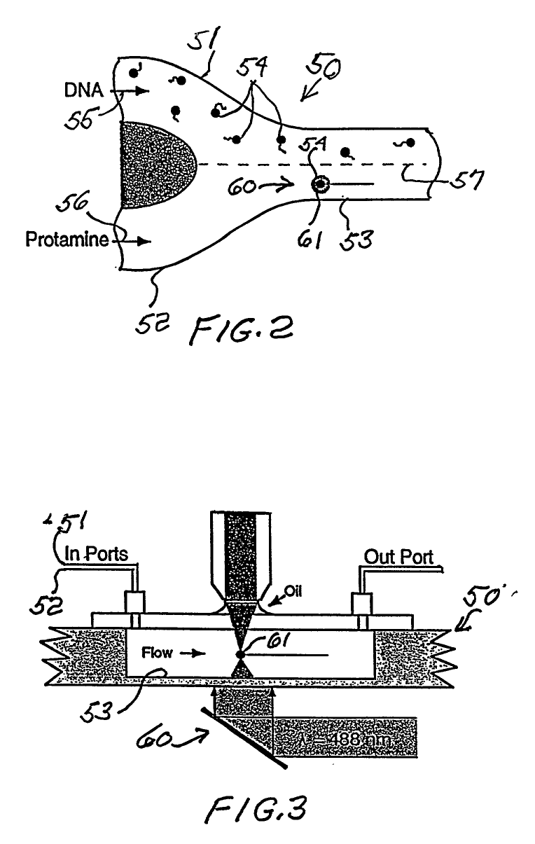

Experiments with our dual-port flow cells (see FIGS. 2-5) have shown that little mixing takes place between the different chemical species in the flow cell. The distance of radial diffusion is given by:

<r²>=6Dt

where the radial diffusion constant D=(kT)/6πη, k is Boltzmann's constant, T is temperature in degrees Kelvin, η is the viscosity of the chemical species, a is the molecular radius=(m/ρ)^{1/3}, m is the molecular mass, ρ the density of the chemical species, and t is the time. The buffer typically used in our experiments contains 50% sucrose (η=15.3 cp). The sucrose is used because it is viscous and allows the 1 um spheres to be suspended in liquid for a long time as well as damping the Brownian motion of the beads and making them easier to trap. For protamine the molecular radius a=89 nm. For t=30 seconds, the radial diffusion r=5.4 um, in approximate agreement with our experimental observations for a dual port flow cell.

Experimental verification of the invention is described generally hereinafter with respect to FIGS. 2-5, described in detail in an article by L. R. Brewer et al, “Protamine-Induced Condensation and Decondensation of the Same DNA Molecule”, Science, Vol. 286, Oct. 1, 1999, pp. 120-123, and in an article by J. Felton et al, “Biophysical Analysis of DNA-Protein Interactions Using an Optical Trap to Manipulate Single DNA Molecules”, Laboratory Directed Research & Development, FY 1999, p. 3-18, each incorporated herein by reference thereto.

In the experimental verification, Lambda-phage DNA concatemers (20 to 80 μm long) were tagged at one end with a biotinylated oligonucleotide attached to a 1 -μm streptavidin-coated polystyrene bead and stained with the intercalating dye YOYO-1. These molecules were introduced through one port of a bifurcated flow cell (see FIG. 2) and the condensing agent protamine (or Arg₆) through another port so that the two solutions flow side by side with minimal mixing.

As seen in FIG. 2, the flow cell generally indicated at 50 includes a pair of input channel sections 51 and 52 and a common channel section 53. DNA molecules 54 are directed through channel section 51 into channel 53, as indicated by arrow 55, while protamine is directed through channel section 52 into channel 53, as indicated by arrow 56. The two solutions 55 and 56 flow side by side as indicated by dashed line 57 with minimal mixing.

An infrared optical trap (see FIG. 3) was used to move an individual DNA molecule 54, via its attached bead, from the sample (DNA) side 51 to the condensing agent (protamine or protein) side 52 of the flow cell 50.

As shown in FIG. 3, an optical trap generally indicated at 60 is operatively mounted to common channel 53 of flow cell 50 of FIG. 2. The optical trap is shown holding a bead 61 of a DNA molecule 54 in the condensation side 52 containing the protamine solution 56, as seen in FIG. 2. By way of example, the bead 61 is 1 μm and the wavelength of the infrared optical trap is 488 nm. Since optical traps are known in the art, further description thereof is deemed unnecessary to provide an understanding of the invention.

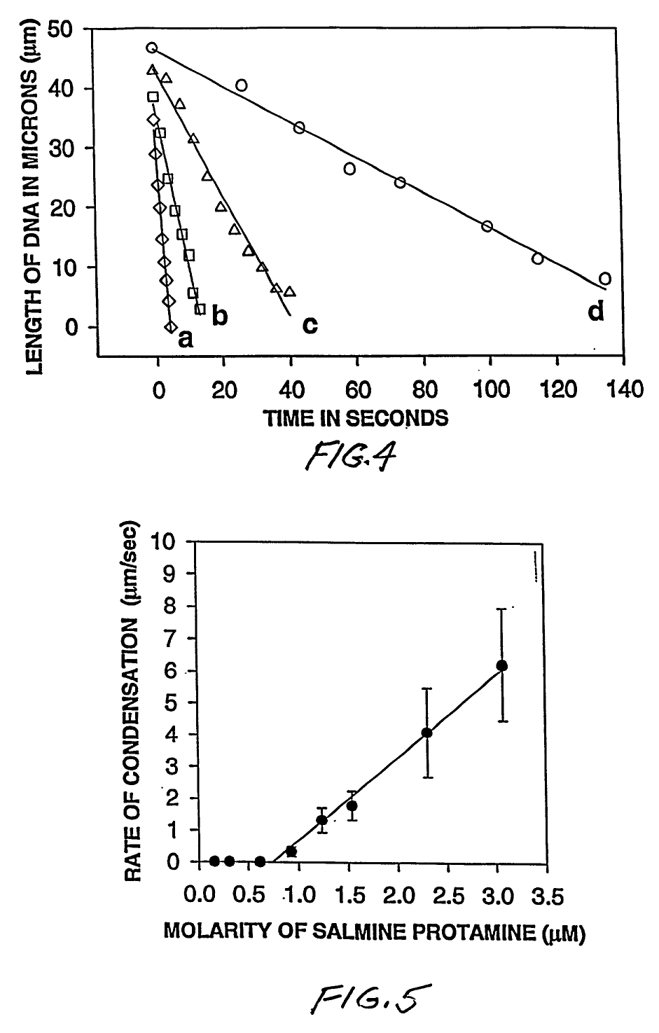

The change in length verses time for four different DNA molecules, indicated at a, b, c&d, as they condensed in different concentrations of protamine is shown in FIG. 4. The tests were conducted with a flow speed, v=50 μm/s, with a being 3.1 μM, b being 1.6 μm, c being 1.2 μm, and d being 0.93 μM.

Experiments conducted at different protamine concentrations showed that the rate of condensation was limited by the rate of protamine binding to the DNA molecule. The change in rate, see FIG. 5, was linear, with a slope of 2.6±0.47 μm/μM-s. This corresponds to a rate of protamine binding to DNA of 600±110 molecules/μM-s. The rate of condensation was measured at two different concentrations of YOYO-1 (0.1 and 0.02 μM) to determine whether intercalated YOYO-1 molecules affect the condensation rate. No statistically significant difference in the rates was observed. The condensation rates of FIG. 5 were determined by collecting data for about 200 individual DNA molecules condensed by protamine. For further details of the invention verification experiments reference to the above-cited article by L. R. Brewer et al should be made.

It has thus been shown that the present invention provides the ability to introduce an individual DNA molecule to proteins or a variety of different chemical environments both at a precise time, and sequentially. By the use of the multichannel flow cell and an optical trap a single DNA molecule may be pulled into each of a variety of different chemical species sequentially, and the resultant change in the structure of the DNA molecule can be observed using fluorescence microscopy.

While particular embodiments of the flow cell have been illustrated and described along with particular materials and parameters to exemplify and teach the principles of the invention, such are not intended to be limiting. Modifications and changes may become apparent to those skilled in the art, and it is intended that the invention be limited only by the scope of the appended claims.

The invention claimed is

1. In a device for manipulation of molecules in a laminar flow, the improvement comprising:
a multichannel flow cell in combination with an optical trap.

2. The improvement of claim 1, wherein said multichannel flow cell has an inlet port for each channel.

3. The improvement of claim 2, wherein said multichannel flow cell includes at least three channels whereby three difference solutions may be introduced into the channel.

4. The improvement of claim 3, wherein a molecule in a first channel is pulled sequentially into at least two additional channels by said optical trap.

5. The improvement of claim 1, wherein said multichannel flow cell includes a plurality of inlet channel sections terminating in a common channel section having an outlet.

6. The improvement of claim 5, wherein each of said plurality of inlet channel sections are connected to an inlet opening.

7. The improvement of claim 6, wherein each said inlet opening is connected to an inlet port.

8. The improvement of claim 1, wherein said multichannel flow cell comprises:
a first plate having fluidic channels formed therein, and
a second plate having a plurality of openings aligned with said fluidic channels,
said fluid channels in said first plate comprising a plurality of inlet channel sections each terminating in a common channel section.

9. The improvement of claim 8, wherein said optical is located adjacent said common channel section.

10. The improvement of claim 9, wherein said plurality of openings in said second plate are aligned with said inlet channel sections and with an end section of said common channel section.

11. The improvement of claim 10, wherein said end section has a tapering configuration.

12. The improvement of claim 8, wherein each of said plurality of inlet channels sections terminate in said common channel section in a parallel configuration.

13. The improvement of claim 12, wherein said plurality of inlet channel sections are of a number greater than two, and wherein said optical trap is operatively mounted to move a molecule sequentially from one inlet channel section to an adjacent inlet channel section.

14. The improvement of claim 13, wherein a first of said inlet channel sections is provided with DNA molecules with attached beads, and wherein each other of said inlet channel sections is provided with proteins or different chemical species, whereby movement of an individual molecule by said optical trap through said common channel section from inlet channel section termination area to inlet channel section termination area sequentially enables observation of changes in the structure of the DNA molecule.

15. In an apparatus utilizing an optical trap to enable studies that characterize the binding of proteins to DNA, the improvement comprising:
a multichannel flow cell for interacting single, optically trapped, DNA molecules with different chemical species sequentially,
whereby the resultant change in the structure of the DNA molecule can be observed using fluorescence microscopy.

16. The improvement of claim 15, wherein said multichannel flow cell includes a number of separate fluidic input channels having parallel end sections which terminate in one end of a common fluidic channel having an output at the opposite end, whereby fluids passing from said input channels through said common channel flow in a side by side relation with little mixing, and wherein said optical trap is located adjacent said common channel, whereby molecules passing from a first of said input channels into said common channel can be sequentially moved through different chemical species passing from the remaining input channels through said common channel.

17. The improvement of claim 15, wherein said multichannel flow cell comprises:
a first plate,
a second plate,
said first plate having a number of inlet channels which terminals in a common channel,
said common channel having an outlet,
said inlet channels having parallel end sections which terminate in said common channel,
said second plate having a number of opening therein, said openings being positioned to align with said inlet channels and said outlet of said common channel, and
said optical trap being operably mounted adjacent said common channel.

18. A multichannel flow cell for interacting single, optically trapped, DNA molecules with different chemical species, comprising:
a number of separate inlet channels each having a parallel end sections, and
a common channel connected at one end to said parallel end sections and having an output at an opposite end,
whereby fluids directed through said inlet channels and discharging from said parallel end sections into said common channel pass along said common channel with little mixing.

19. The multichannel flow channel of claim 18, wherein said inlet channels and said common channel are located in a surface of a first member, and wherein inlets to said inlet channels and an outlet for said output of said common channel are located in a second member.
